(19) United States
(12) Patent Application Publication
(10) Pub. No.: US 2025/0303019 A1
(43) Pub. Date: Oct. 2, 2025

(54) BIODEGRADABLE CEMENT COMPOSITIONS INCLUDING LINEAR CYCLIC POLYANHYDRIDES

(71) Applicants: Devon A. Shipp, Potsdam, NY (US); Damien Samways, Potsdam, NY (US); Philip Yuya, Potsdam, NY (US); Ponsubha Sivabalan, Potsdam, NY (US)

(72) Inventors: Devon A. Shipp, Potsdam, NY (US); Damien Samways, Potsdam, NY (US); Philip Yuya, Potsdam, NY (US); Ponsubha Sivabalan, Potsdam, NY (US)

(73) Assignee: Clarkson University, Potsdam, NY (US)

(21) Appl. No.: 19/091,471

(22) Filed: Mar. 26, 2025

Related U.S. Application Data
(60) Provisional application No. 63/570,005, filed on Mar. 26, 2024.

(51) Int. Cl. A61L 24/06; A61L 24/00; C08F 20/14

(52) U.S. Cl. CPC A61L 24/06; A61L 24/0042; C08F 20/14; A61L 2430/02

(57) ABSTRACT

A class of biodegradable, surface-eroding cyclic poly(methacrylic anhydride) (PMAA) polymer-based biomaterials that are useful as bone cement or bio-adhesives. The synthesis of the biomaterials involves the preparation of linear cyclic PMAA prepolymers. The biomaterials may be further processed to make bone cement for orthopedic applications. Extensive testing of the biomaterials has confirmed that they are biodegradable, have a peak exotherm that is below 45° C., have no or negligible shrinkage, and have good mechanical properties. The biomaterials therefore provide advantages over commercial poly(methyl methacrylate) (PMMA) based bone cements that are currently used in medical applications ranging from dentistry to orthopedics.

### CROSS-REFERENCE TO RELATED APPLICATIONS

The present application claims priority to U.S. Provisional Application No. 63/570,005 filed on Mar. 26, 2024, hereby incorporated by reference in its entirety.

### BACKGROUND OF THE INVENTION

### 1. Field of the Invention

The present disclosure relates to biomaterials and, more particularly to cyclic poly(methacrylic anhydride) (PMAA) polymers that can be used to form biodegradable implantable biomaterials for orthopedics, dentistry, cementoplasty, vertebroplasty, or kyphoplasty applications.

### 2. Description of the Related Art

In the last 50 years, the use of polymer-based cements for joint replacements and other surgical procedures has become standard practice. In particular, conventional poly(methacrylic methacrylate) (PMMA) based bone cements serve a variety of purposes, including anchoring an implant in place, restoring surgeries of the skull, joining vertebrae, distributing stresses between the rigid metallic implant and the bone, and in some cases, preventing infection by releasing antibiotics.

Conventional polymer-based bone cements are generally provided with two separate components, a liquid component and a powder component. The liquid component of bone cement is usually a liquid monomer, primarily methyl methacrylate, along with an initiating system. The powder component is usually a dry powder mixture with a primary constituent that is a homopolymer or copolymer of methyl methacrylate. The bone cement may also contain additives like zirconium dioxide, that will provide contrast once the cement cures and the area of the body covered by the cement is x-rayed. Bone cement is inserted in the body, usually to join natural or artificial bone portions, and is cured (hardened) by polymerization of the monomer. Redox catalyst systems are used which comprise an organic peroxy compound, usually dibenzoyl peroxide (BPO) as an initiator, with a reducing agent (accelerator), e.g., N,N-dimethyl-p-toluidine (DMT).

PMMA based cements have several disadvantages, such as non-biocompatibility, non-biodegradability, and tissue adverse effects when associated with long-term implantation. The disadvantages can limit the use of PMMA bone cement. For example, as the cement preparation involves exothermic radical polymerization, it is not a 100 percent efficient process (i.e., does not consume all monomer double bonds) and thus results in unreacted monomers that are capable of leaching into the surrounding tissue. In addition, temperatures have been recorded to reach 47° C. at the bone cement interface, thereby inducing necrosis of osteocytes during the several minutes of polymerization time. Unreacted methyl methacrylate monomer is toxic to cells, which results in apoptosis in tissue and has the potential for adverse allergic reactions. Methyl methacrylate has also been attributed to bone cement implantation syndrome (BCIS), which is characterized by cardiovascular and pulmonary complications such as hypoxia, hypotension, increased pulmonary vascular resistance, and cardiac arrest.

When compared to conventional PMMA cement, the use of biodegradable bone cement offers a number of advantages. For example, a degradable cement may promote greater osseointegration than standard cement, leading to a more secure prosthesis. Current approaches for forming biodegradable bone cement are discussed in U.S. Pat. Nos. 6,685,928, 5,010,167, and 5,902,599. For example, U.S. Pat. No. 5,902,599 discloses a composite material having a surface-eroding synthetic matrix based upon cross-linked polyanhydrides. These approaches do not necessarily provide polymeric materials that remain mechanically strong in situ during the degradation process and often have toxic byproducts during degradation.

As a result, there is a need in the art for biodegradable polymers for use in applications such as dentistry and orthopedics and methods are needed for producing mechanically strong, biodegradable polymeric implants that can be easily implanted, conformed to a specified purpose, potentially delivery drugs and therapeutics, and polymerized in vivo.

### BRIEF SUMMARY OF THE INVENTION

The present invention provides biodegradable polymers having optimal mechanical properties for dental and orthopedic applications even as the polymers degrade. As an example, the present invention includes functional cyclopolymers of methacrylic anhydride with biodegradability that address the shortcomings of traditional PMMA-based bone cements. The present invention further provides bone cement by utilizing surface-eroding PMAA cyclopolymers to provide bone-like mechanical strength throughout the degradation process. Cements according to the present invention initially provide mechanical and structural stability and then degrade via surface erosion in a controlled manner while promoting bone ingrowth. Bone cement formulations using methacrylic anhydride cyclopolymers according to the present invention undergo hydrolytic bond cleavage, which leads to water soluble and nontoxic acid end-products. As a result, bone cements according to the present invention avoid surgical removal of the system.

In one example, the present invention may be a composition having a liquid component including a monomer selected from the group consisting of methyl methacrylate, n-butyl methacrylate, tert-butyl methacrylate, n-butyl acrylate, ethylene glycol diacrylate, ethylene glycol dimethacrylate, poly(ethylene glycol dimethacrylate), and other (meth)acrylic monomers, and a powder component including a linear cyclic polyanhydride. The linear cyclic polyanhydride may be cyclic poly(methacrylic anhydride). The powder component may further comprise a copolymer selected from the group consisting of poly(methacrylic anhydride-co-methyl methacrylate), poly(methacrylic anhydride-co-n-butyl methyl methacrylate), poly(methacrylic anhydride-co-tert-butyl methyl methacrylate), and poly(methacrylic anhydride-co-n-butyl acrylate). The liquid component may further comprise a crosslinking monomer. The crosslinking monomer may be selected from the group consisting of ethylene glycol diacrylate, ethylene glycol dimethacrylate, poly(ethylene glycol dimethacrylate), and other di(meth)acrylic monomers. The liquid component may further comprise a non-crosslinking monomer. The non-crosslinking monomer may be selected from the group consisting of methyl methacrylate, n-butyl methacrylate, tert-butyl methacrylate, n-butyl acrylate, and other (meth)acrylates. The liquid component may be methacrylic anhydride. The liquid component may comprise between 5 and 60 percent by weight of a comonomer selected from the group consisting of methyl methacrylate, n-butyl methacrylate, tert-butyl methacrylate, n-butyl acrylate, ethylene glycol diacrylate, ethylene glycol dimethacrylate, and poly(ethylene glycol dimethacrylate). The liquid component may include an activator. The power component may include at least one of a redox-initiator and a photo-initiator. The composition may be formed into a biodegradable implant.

In another example, the present invention may be a method of forming a cyclopolymer by providing a divinyl anhydride monomer and then cyclopolymerizing the divinyl anhydride monomer in the presence of an initiator, a chain transfer agent, and a solvent to form a linear cyclic anhydride. The divinyl anhydride monomer may be methacrylic anhydride. The step of cyclopolymerizing the divinyl anhydride monomer may include copolymerizing the divinyl anhydride monomer with a (meth)acrylate monomer. The (meth)acrylate monomer may be selected from the group consisting of methyl methacrylate, n-butyl methacrylate, tert-butyl methacrylate, n-butyl acrylate, and other (meth)acrylates. The step of cyclopolymerizing the divinyl anhydride monomer may be performed using a radical solution or bulk polymerization. The chain transfer agent may be (N,N′,N″,N′″-tetrafluoro-diborato)bis[μ-(2,3)-butanedionedioximato)]cobalt (II)). The solvent may be at least one of cyclohexanone and benzonitrile. The step of cyclopolymerizing the divinyl anhydride monomer may be performed at a temperature between 50 and 90 degrees Celsius.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of the radical cyclopolymerization of MAA with an appropriate CTA to provide unsaturated end groups according to the present invention.

FIG. 2 is a series of graphs of (a) FT-IR, (b) ¹H NMR, and (c) ¹³C NMR spectra of PMAA prepared with CoBF under highly diluted conditions (MAA=0.27 M), and (d) the GPC curve of PMAA, in accordance with an embodiment of the invention

FIG. 3 is a plot of normalized mass erosion as a function of time for each composition tested, in accordance with an embodiment of the invention.

FIG. 4 is a series of images of degradation as a function of time for each composition tested according to the present invention.

### DETAILED DESCRIPTION OF THE INVENTION

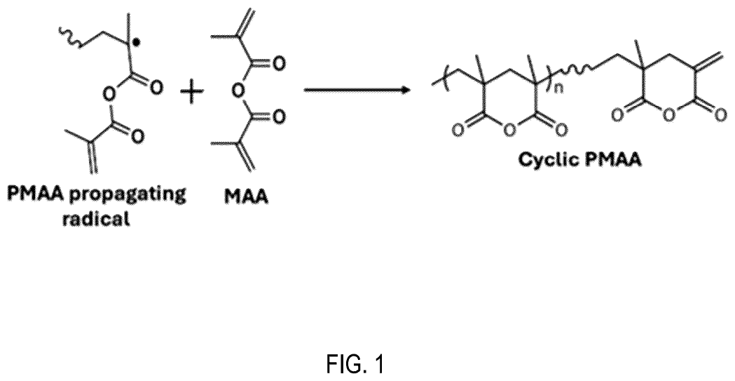

Referring to the figures, wherein like numerals refer to like parts throughout, there is seen in FIG. 1 the radical polymerization of methacrylic anhydride (MAA) to provide a linear cyclic anhydride polymer, cyclic poly(methacrylic anhydride) (PMAA) that are soluble and still contain the reactive anhydride group in the polymer backbone. The linear cyclic anhydride polymer can undergo hydrolytic bond cleavage, leading to water soluble and nontoxic acid end-products. The linear cyclic anhydride polymer is thus useful as the powder component of a cement composition employing a liquid component and a powder component that are mixed and polymerized in situ, such as in orthopedics, dentistry, cementoplasty, vertebroplasty, or kyphoplasty applications.

To achieve the goal of making linear cyclopolymers based on MAA that are soluble and still contain the reactive anhydride group in the polymer backbone, the initial cyclopolymerization of MAA was performed under highly diluted conditions. Cyclopolymerization of MAA by conventional radical polymerization using an effective chain transfer agent (CTA), such as N,N′,N″,N″-tetrafluoro-diborato)bis[μ-(2,3)-butanedionedioximato)]cobalt (II)) (CoBF) and radical initiator, such as 1,1-azobis(cyclohexanecarbonitrile) (ABCN), allows high conversion synthesis of fully soluble PMAA cyclopolymer, as seen in FIG. 1. The peak at 1635 cm⁻¹ in spectrum (a) depicts the unsaturated end group of each polymer chain made with CoBF. The signals related to the vinyl end group are present at 6.01 and 6.44 ppm in spectrum (b). The peak at 2.5 (★) ppm in spectrum (b) and 39.96 (★) ppm in spectrum (c) are attributed to the solvent DMSO-d6, and the peak at 3.3 (♦) ppm is presumed to arise from the water. In (d), the GPC curve of PMAA is shown. DMSO was used as the eluent with a flow rate of 1 ml/min, and the total elution time was set at 15 minutes.

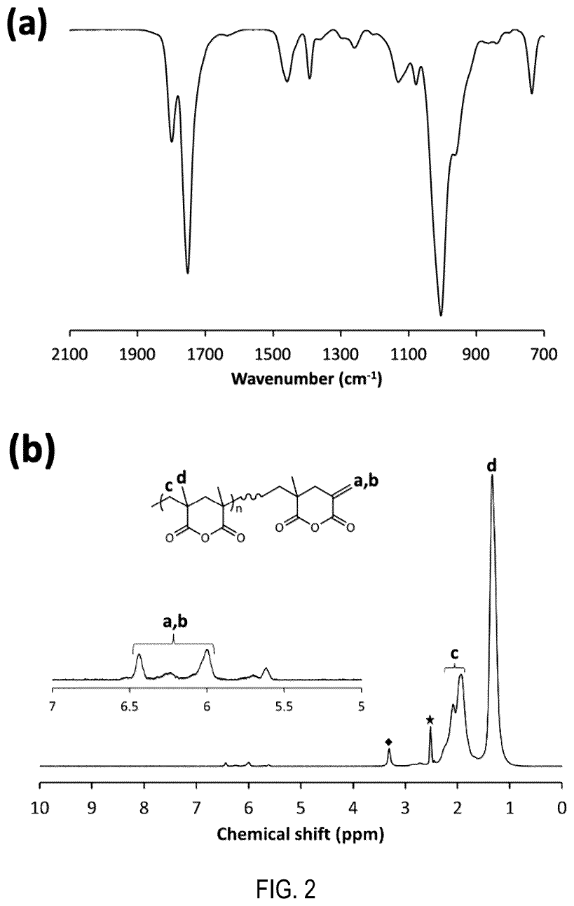

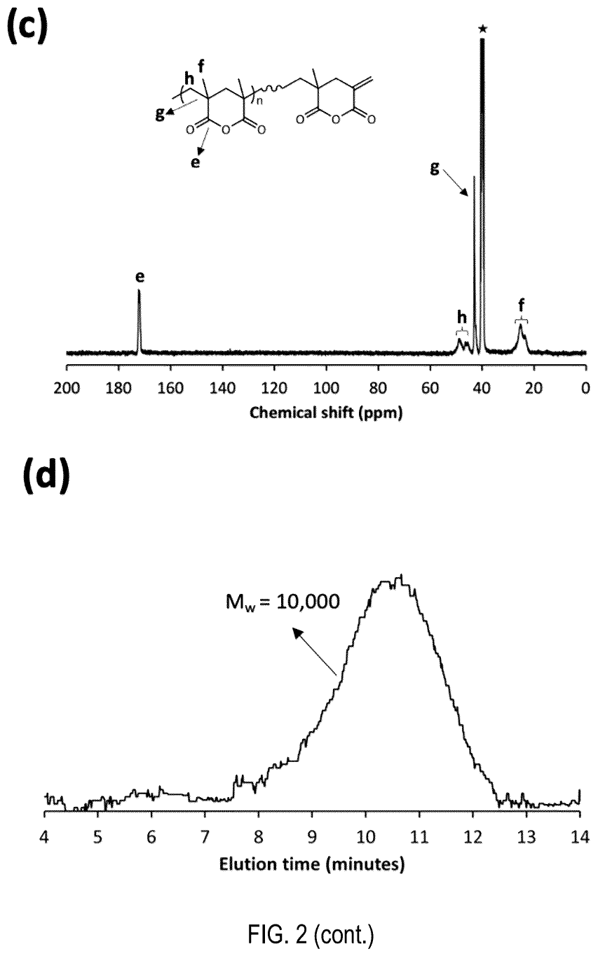

The PMAA cyclopolymer was prepared under highly diluted conditions (Table 1—sample PMAA-1) and confirmed by Fourier Transform Infrared (FT-IR) and 1H and 13C nuclear magnetic resonance (NMR) analyses. The spectra of FT-IR and NMR are shown in FIG. 2(a-c). The weight average molecular weight (Mw) of PMAA was measured using gel permeation chromatography (GPC) and found to be 10,000, as seen in FIG. 1(d). This value was obtained by comparison with commercial PVP polymers purchased from Sigma-Aldrich. Additionally, the glass transition temperature (Tg) of PMAA ascertained using differential scanning calorimetry (DSC), is recorded at 117° C.

Soluble PMAA was attainable even at moderate MAA concentrations with no crosslinking in the presence of CoBF. This was achieved by studying various MAA concentrations and temperatures with and without CoBF and examining the impact of these parameters on the PMAA microstructure. Importantly, the non-diluted MAA polymerization in the presence of CoBF led to the production of cyclic six-membered soluble polymers. On the other hand, crosslinked PMAA resulted from the polymerization of MAA at and above monomer concentrations of 3.4 M in the absence of CoBF.

Radical copolymerization of MAA by reactive blending in different feed ratios of comonomers, starting from methyl methacrylate (MMA), n-butyl methacrylate (n-BMA), tert-butyl methacrylate (t-BMA), and n-butyl acrylate (n-BA) was explored. Additionally, by employing CoBF as a CTA in radical copolymerization, the crosslinking of MAA was reduced by reducing the molecular weight. The formation of copolymers was confirmed by FT-IR and NMR characterization. The prepared copolymers showed better solubility in various organic solvents such as tetrahydrofuran (THF), dimethylformamide (DMF), and dimethyl sulfoxide (DMSO) and had a range of glass transition temperatures (70-150° C.) and weight average molecular weights (Mw=3,000-7,00,000).

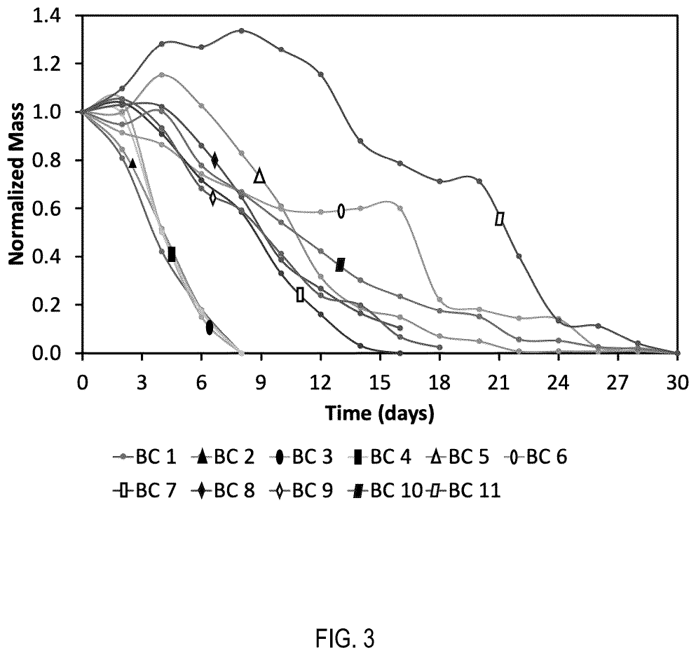

The mass erosion profiles of bone cement (BC) based on PMAA are shown in FIG. 3. PMAA homopolymers and copolymers made with MMA, n-BMA, and t-BMA quickly degraded in less than 10 days. Since the hydrolysis of the anhydride is the primary factor influencing the erosion rate, the kind of comonomer has less of an effect at higher MAA concentrations.

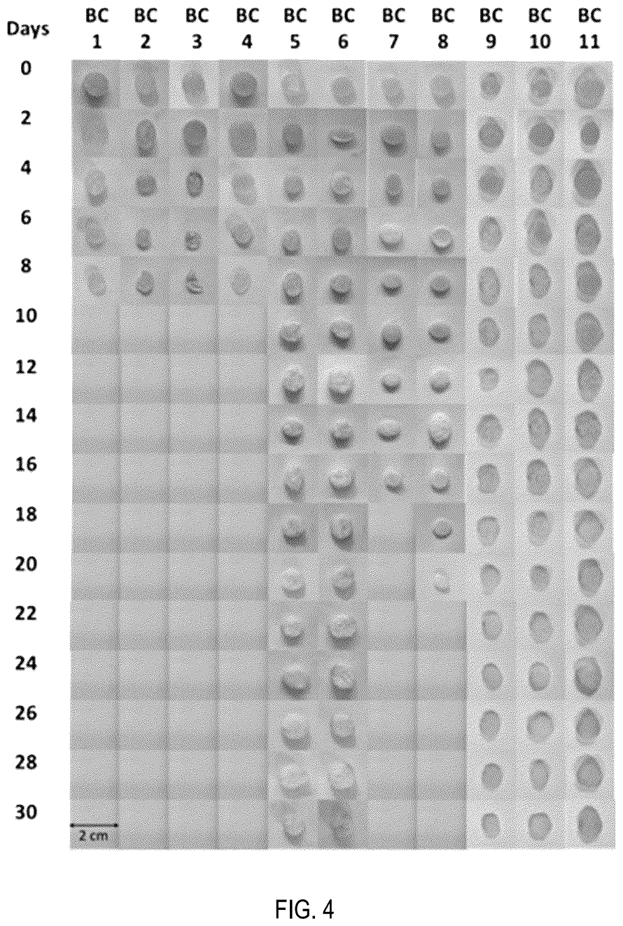

Surface erosion was qualitatively observed through photographs during the degradation process in FIG. 4. All discs retained the shape throughout the degradation process, thus qualitatively confirming surface erosion. Compared to other BCs, those produced using EGDMA and PEGDMA as a comonomer experienced gradual degradation and surface erosion and lasted for more than 20 days. The degradation time of these cements was tuned according to the different percentages of monomers. Based on nanoindentation tests, the calculated hardness and elastic modulus of BCs ranged from 90 to 350 MPa and 1.4 to 7 GPA respectively.

### Example 1

### Cyclic Poly(Methacrylic Anhydride) (PMAA) Preparation

Cyclic PMAA was synthesized by using radical polymerization. The given amounts (sample PMAA-1 in Table 1 below) of reagents were placed in a round-bottom flask, the reagents were degassed by bubbling with N2 for 30 min, and then the flask was placed in a preheated oil bath equipped with a magnetic stirrer. The polymerizations were conducted at 90° C. for 24 hours. The polymer was recovered by precipitation in diethyl ether, and purified by repeated washing with diethyl ether and drying for 24 h in a vacuum oven at 40° C. In addition, the same procedure was used to synthesize a series of polymers prepared by varying the concentrations of monomer and chain transfer agent (samples PMAA-2-PMAA-13 in Table 1). The solubility of the synthesized PMAA under various reaction conditions was tested in DMF and DMSO. About 0.1 g of the polymer was added to 1 ml of solvent in a glass vial and kept overnight. The solubility behavior of each polymer was ascertained by visual inspection and assessment of the back pressure generated by pushing the solution through a 0.22 μm polytetrafluoroethylene (PTFE) filter using a hand syringe.

TABLE 1
Reaction conditions^{a} used for MAA radical polymerizations.

| | | | | | | Solubility in |
|---|---|---|---|---|---|---|
| | MAA | | | | Time | DMF & |
| Samples | (M) | CoBF | | ABCN | (h) | DMSO |
| PMAA-1 | 0.27 | 30.0 | μM | 0.19 mM | 24 | Yes |
| PMAA-2 | 1.00 | 30.0 | μM | 0.70 mM | 24 | Yes |
| PMAA-3 | 3.40 | 30.0 | μM | 2.40 mM | 1.5 | Yes |
| PMAA-4 | 6.27 | 30.0 | μM | 4.80 mM | 1.0 | Yes |
| PMAA-5 | 6.71^{b} | 0.78 | mM | 4.10 mM | 1.0 | Yes |
| PMAA-6^{c} | 6.71^{b} | 0.78 | mM | 4.10 mM | 1.0 | No |
| PMAA-7 | 0.27 | — | | 0.19 mM | 24 | Yes |
| PMAA-8 | 1.00 | — | | 0.70 mM | 24 | Yes |
| PMAA-9 | 2.80 | — | | 1.90 mM | 2.0 | Yes |
| PMAA-10 | 3.40 | — | | 2.40 mM | 1.0 | No |
| PMAA-11 | 0.27 | 3.00 | μM | 0.19 mM | 24 | Yes |
| PMAA-12 | 0.27 | 7.50 | μM | 0.19 mM | 24 | Yes |
| PMAA-13 | 0.27 | 15.0 | μM | 0.19 mM | 24 | Yes |

^{a}Reactions thermally initiated (90° C.) with cyclohexanone as solvent except as noted. Typical reaction volume = 24 ml.
^{b}Reactions performed bulk (no solvent).
^{c}Reaction initiated using UV lamp at 20° C.

### Example 2

### Copolymer Preparation

Copolymers (CPs) were synthesized by using radical polymerization. The given amounts (Table 2) of reagents were placed in a round-bottom flask, the reagents were degassed by bubbling with N2 for 30 min, and then the flask was placed in a preheated oil bath equipped with a magnetic stirrer. The polymerizations were conducted at 90° C. for 24 hours. The polymer was recovered by precipitation in diethyl ether, and purified by repeated washing with diethyl ether and drying for 24 h in a vacuum oven at 40° C. In addition, all of the reactions that are reported in Table 2 were carried out in a different series without the use of a chain transfer agent. The solubility of the synthesized copolymers was tested in THF, DMF, and DMSO. About 0.1 g of the polymer was added to 1 ml of solvent in a glass vial and kept overnight. The solubility behavior of each polymer was ascertained by visual inspection and assessment of the back pressure generated by pushing the solution through a 0.22 μm PTFE filter using a hand syringe.

TABLE 2
Reaction conditions^{a} used for comonomer radical polymerizations.

| | MAA | MMA | n-BMA | t-BMA | n-BA | CoBF |
|---|---|---|---|---|---|---|
| Samples | (M) | (M) | (M) | (M) | (M) | (μM) |
| CP-1 | 0.2025 | 0.0675 | — | — | — | 30.0 |
| CP-2 | 0.135 | 0.135 | — | — | — | 30.0 |
| CP-3 | 0.0675 | 0.2025 | — | — | — | 30.0 |
| CP-4 | 0.2025 | — | 0.0675 | — | — | 7.50 |
| CP-5 | 0.135 | — | 0.135 | — | — | 7.50 |
| CP-6 | 0.0675 | — | 0.2025 | — | — | 7.50 |
| CP-7 | 0.2025 | — | — | 0.0675 | — | 30.0 |
| CP-8 | 0.135 | — | — | 0.135 | — | 30.0 |
| CP-9 | 0.2025 | — | — | — | 0.0675 | 30.0 |
| CP-10 | 0.135 | — | — | — | 0.135 | 30.0 |
| CP-11 | 0.0675 | — | — | — | 0.2025 | 30.0 |

^{a}Reactions thermally initiated (90° C.) with cyclohexanone as solvent. Typical reaction volume = 24 ml.

### Example 3

### Bone Cement (BC) Formulation

To make a bone cement, a 1:1 solid (polymer & BPO)-to-liquid (monomer & DMT) ratio was used. In the cement formulations, 1 wt. % of BPO initiator, and 3 wt. % of DMT activators were used regarding monomer concentration. The BPO initiator was first mixed with a homopolymer or copolymer, and the DMT was dissolved in a monomer or comonomer, and then the liquid mixture was introduced to the solid mixture and mixed well, which was then transferred to the Teflon mold for hardening. Following is a list of the various cement compositions.

$$BC 1 = [ P M A A + M AA ]$$

$$BC 2 = [ P ( M A A + M M A ) + M A A ]$$

$$BC 3 = [ P ( M A A + n - B M A ) + M A A ]$$

$$BC 4 = [ P ( M A A + t - B M A ) + M A A ]$$

$$BC 5 = [ PMAA + ( MAA + EGDMA - 90 + 10 wt . \% ) ]$$

$$BC 6 = [ PMAA + ( MAA + EGDMA - 75 + 25 wt . \% ) ]$$

$$BC 7 = [ PMAA + ( MAA + PEGDMA - 90 + 10 wt . \% ) ]$$

$$BC 8 = [ PMAA + ( MAA + PEGDMA - 75 + 25 wt . \% ) ]$$

$$BC 9 = [ PMAA + ( MAA + PEGDMA - 60 + 40 wt . \% ) ]$$

$$BC 10 = [ PMAA + ( MAA + PEGDMA - 50 + 50 wt . \% ) ]$$

$$BC 11 = [ PMAA + ( MAA + PEGDMA - 40 + 60 wt . \% ) ]$$

### Example 4

### Degradation Study of Bone Cements

In vitro degradation study was done in PBS (7.4 pH) at 37° C. The BC disk volume and mass were measured with a caliper and 4-digit balance respectively before placement into 50 mL of 1×PBS 7.4 pH buffer in a glass container. The glass containers were then placed into a water bath at 37° C. to monitor the mass and volume loss as a function of time. PBS 1×buffer at 7.4 pH and 37° C. were selected to mimic physiological conditions. The BC samples were removed from the PBS buffer daily and dabbed carefully with a lint-free tissue to record the mass and volume. The PBS buffer was replaced daily to maintain the pH due to acid production. In two-day intervals, pictures of the disks were taken before they started to degrade on day zero.

### Example 5

### Mechanical Study of Bone Cement

Nanoindentation testing was conducted to obtain comprehensive information about the BCs local mechanical properties, like hardness and the elastic modulus. The testing was performed on the polished surface of the BC samples using a nanoindenter (TI-950 Hysitron Triboindenter, Bruker Co., Billerica, MA, USA). A standard diamond Berkovich tip (three-faceted pyramid) with a total included angle of 142.3°, half-angle of 65.35°, and radius of curvature of 100 nm was used as the indenter tip.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, embodiments may be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

What is claimed is:

1. A composition, comprising:
a liquid component including a monomer is selected from the group consisting of methyl methacrylate, n-butyl methacrylate, tert-butyl methacrylate, n-butyl acrylate, ethylene glycol diacrylate, ethylene glycol dimethacrylate, and poly(ethylene glycol dimethacrylate); and
a powder component including a linear cyclic anhydride.

2. The composition of claim 1, wherein the linear cyclic anhydride is cyclic poly(methacrylic anhydride).

3. The composition of claim 1, wherein the powder component further comprises a copolymer selected from the group consisting of poly(methacrylic anhydride-co-methyl methacrylate), poly(methacrylic anhydride-co-n-butyl methyl methacrylate), poly(methacrylic anhydride-co-tert-butyl methyl methacrylate), and poly(methacrylic anhydride-co-n-butyl acrylate).

4. The composition of claim 1, wherein the liquid component further comprises a crosslinking monomer.

5. The composition of claim 4, where the crosslinking monomer is selected from the group consisting of ethylene glycol diacrylate, ethylene glycol dimethacrylate, and poly(ethylene glycol dimethacrylate).

6. The composition of claim 1, wherein the liquid component further comprises a non-crosslinking monomer.

7. The composition of claim 6, where the non-crosslinking monomer is selected from the group consisting of methyl methacrylate, n-butyl methacrylate, tert-butyl methacrylate, and n-butyl acrylate.

8. The composition of claim 1, wherein the liquid component consists of methacrylic anhydride.

9. The composition of claim 1, wherein the liquid component comprises between 5 and 60 percent by weight of a comonomer selected from the group consisting of methyl methacrylate, n-butyl methacrylate, tert-butyl methacrylate, n-butyl acrylate, ethylene glycol diacrylate, ethylene glycol dimethacrylate, and poly(ethylene glycol dimethacrylate).

10. The composition of claim 1, wherein the liquid component includes an activator.

11. The composition of claim 1, wherein the powder component includes at least one of a redox-initiator and a photo-initiator.

12. A biodegradable implant formed by the composition of claim 1.

13. A method of forming a cyclopolymer, comprising the steps of:
providing a divinyl anhydride monomer; and
cyclopolymerizing the divinyl anhydride monomer in the presence of an initiator, a chain transfer agent, and a solvent to form a linear cyclic anhydride.

14. The method of claim 13, wherein the divinyl anhydride monomer is methacrylic anhydride.

15. The method of claim 13, wherein the step of cyclopolymerizing the divinyl anhydride monomer includes copolymerizing the divinyl anhydride monomer with an acrylate monomer.

16. The method of claim 15, wherein the acrylate monomer is selected from the group consisting of methyl methacrylate, n-butyl methacrylate, tert-butyl methacrylate, and n-butyl acrylate.

17. The method of claim 13, wherein the step of cyclopolymerizing the divinyl anhydride monomer is performed using at least one of radical solution and bulk polymerization.

18. The method of claim 13, wherein the chain transfer agent is (N,N′,N″,N′″-tetrafluoro-diborato)bis[μ-(2,3)-butanedionedioximato)]cobalt (II)).

19. The method of claim 13, wherein the solvent is at least one of cyclohexanone and benzonitrile.

20. The method of claim 13, wherein the step of cyclopolymerizing the divinyl anhydride monomer is performed at a temperature between 50 and 90 degrees Celsius.
